(19) United States
(12) Patent Application Publication
(10) Pub. No.: US 2004/0146531 A1
(43) Pub. Date: Jul. 29, 2004

(54) Virus-like particles devoid of HPV 16 type-specific antibody epitopes as carriers of peptides for introduction into cells

(75) Inventors: Per Antonsson, Lund (SE); Karin Kristensson, Lund (SE); Marie Wallen-Ohman, Lund (SE); Joakim Dillner, Danderyd (SE); Peter Lando, Malmo (SE)

Correspondence Address: BURNS DOANE SWECKER & MATHIS L L P, POST OFFICE BOX 1404, ALEXANDRIA, VA, 22313-1404, US

(73) Assignee: Active Biotech AB, Lund (SE)

(21) Appl. No.: 10/759,125

(22) Filed: Jan. 20, 2004

Related U.S. Application Data
(63) Continuation-in-part of application No. 10/048,016, filed on Jan. 28, 2002.
Application No. 10/048,016 is a 371 of international application No. PCT/SE00/01808, filed on Sep. 19, 2000.

(30) Foreign Application Priority Data
Sep. 30, 1999 (SE) 9903534-7

(51) Int. Cl. A61K 39/12; C12N 7/00

(52) U.S. Cl. 424/204.1; 435/235.1

(57) ABSTRACT

The present invention relates to a carrier for introduction of substances into cells comprising a modified major capsid protein L1 of human papillomavirus (HPV-L1 protein) devoid of type-specific epitopes causing production of neutralising antibodies. The invention also includes an oligo- or polynucleotide coding for said carrier, vaccines comprising said carrier or said oligo- or polynucleotide, as well as methods of using the carrier or the oligo- or polynucleotide in vaccination against viral, bacterial or parasite infections as well as against development of certain cancers. Especially, infections of human papillomavirus and the development of cancer as a consequence of such infections are recognised.

This application is a continuation-in-part under 37 C.F.R. § 1.53(b) of U.S. patent application Ser. No. 10/048,016, filed on Jan. 28, 2002, for Vaccine, which was a national stage filing under 37 C.F.R. § 371 of International Application No. PCT/SE00/01808, filed on Sep. 19, 2000.

### FIELD OF THE INVENTION

The present invention relates to a carrier for introduction of substances into cells comprising a modified major capsid protein L1 of human papillomavirus (HPV-L1 protein) devoid of type-specific epitopes causing production of neutralising antibodies. The invention also includes an oligo- or polynucleotide coding for said carrier, vaccines comprising said carrier or said oligo- or polynucleotide, as well as methods of using the carrier or the oligo- or polynucleotide in vaccination against viral, bacterial or parasite infections as well as against development of certain cancers. Especially, infections of human papillomavirus and the development of cancer as a consequence of such infections are recognised.

### BACKGROUND OF THE INVENTION

The Human Papillomavirus (HPV) is long established as the major cause of cervical cancer (1), and in recent years has also been established as a cause of cancers of the penis, vulva, vagina, anus and orofarynx. Indications also exist that the virus may be involved in some cancers of the prostate, esophagus, and in head and neck cancers. HPV vaccine development is therefore a prime priority of preventive cancer research today (2).

There are over 100 types of HPVs. Although types are defined by genetic homology, the genotypes have shown a strikingly good concordance with serotypes, i.e. hyperimmune antisera against one type will only neutralise the same type and not other genotypes. Cross-neutralizations have only been reported for certain closely related types, and have only shown titers two orders of magnitude less than for the type-specific neutralization (2,3).

The HPV capsid consists of 72 capsomers, each containing 5 copies of the HPV major capsid protein L1. A minor capsid protein, L2, is present in much smaller amounts in the capsid (1:12 compared to the L1 protein) and the location of L2 is uncertain (2).

A number of small viruses express capsid protein that, when expressed, self-assemble to form virus-like particles (VLPs) (i.e. particles morphologically similar to virus particles, but lacking the viral genome). The HPV major capsid protein L1 is among the best studied (2). HPV VLPs containing only L1 are morphologically similar to VLPs containing both L1 and L2 (2). Both particles with L1 only and particles with L1/L2 are highly efficient in eliciting a high-titered neutralising antibody response in several animal model systems (rabbits, cows, dogs and rhesus monkeys), even when injected in the absence of adjuvant (2).

Vaccination with papillomavirus VLPs has been shown to be highly efficient for protection, mediated by neutralising antibodies, against subsequent challenge with both cutaneous and mucosal papillomaviruses, but only in a type-specific manner (2). This strong type-specificity is surprising, because the major capsid protein of the HPVs is a highly evolutionarily conserved protein with very few amino acid changes between genetically related, but not cross-neutralising, HPV types.

The most common oncogenic HPVs are HPV16, 18, 31 and 45. HPV16 is found in about 50% of cervical cancers, HPV18 in about 20%, and these four types together correspond to >80% of all cervical cancers. Therefore, a common strategy is to manufacture vaccines containing HPV capsids of the 4 most common HPV types together (2).

Although this strategy may work for achieving significant cancer reduction, it has some distinct disadvantages. The formulation of vaccines containing four active components mixed together involves substantial additional cost in manufacturing, efficacy testing and quality control of each component.

Furthermore, 10-20% of cervical cancers are caused by HPV types not included in the presently manufactured vaccine candidates. Apart from the fact that the vaccine could not possibly protect against these types, the possibility also exists that elimination of the 4 most common oncogenic HPV types may cause an increase in the prevalence of the other oncogenic HPV types, thus further diminishing cancer-preventive gains. This latter scenario is, as predicted from population biology studies, likely to occur if interference exists between different viral types. Several lines of indirect evidence do indicate that interference between HPV types does exist.

Several other HPV types cause significant morbidity and mortality. HPV 6 and 11 cause genital condylomas and recurrent respiratory papillomatoses, and HPVs 5 and 8 cause cutaneous skin-cancers in the immunosuppressed host. In spite of the obvious advantages of broadly cross-reactive vaccines, the possibility of generating a broadly cross-reactive vaccine by modifying the L1 protein to not contain immunodominant type-specific epitopes, has not been proposed. Several surface exposed and cross-reactive epitopes are exposed on papillomavirus particles (WO 96/33737), but are not immunogenic in the presence of the immunodominant type-specific epitope (4). Therefore, by modifying the L1 to remove immunodominant type-specific epitopes, it should be possible to generate a cross-reactive papillomavirus vaccine, using a modified HPV-L1 protein as a carrier of surface exposed HPV derived antibody epitopes.

Furthermore, VLPs are highly efficient in eliciting a cytotoxic T lymphocyte (CTL) response. VLP vaccines have been reported to be highly efficacious (through a CD8+cell-dependent mechanism) in preventing and treating transplantable cancers in several mouse models, in spite of the fact that immunization is made with an exogenous protein (5). The high immunogenicity appears to be due in part to the preservation of an active mechanism for infection of the cell (designated pseudo-infection, as no viral genome is introduced) which results in the capsid protein being processed and presented in the MHC class I presentation pathway (6). VLPs are therefore of general interest from a vaccine biotechnology point of view, because they can be used as a vehicle for efficient immunogenic delivery of any antigen (7).

Efficient immunization using wild-type HPV VLPs carrying foreign antigens has been demonstrated in several systems, e.g. the MAGE melanoma antigens and human immunodeficiency virus antigens.

A potential problem using VLPs as vehicles for immunogenic delivery is blocking by type-specific neutralising antibodies. In Sweden 16% of the adult population are sero-positive for HPV-16, reflecting the importance of the problem.

In addition, therapeutic vaccination is expected to require recurrent treatments, likely to induce a type-specific antibody response towards a wild-type VLP carrier.

Therefore, by modifying the L1 protein to remove type-specific epitopes causing production of neutralising antibodies (8), and by introducing antibody or T-cell epitopes into this carrier, it should be possible to generate an immunological response towards the introduced peptide, without obstruction from type-specific neutralising antibodies directed towards the carrier itself.

### SUMMARY OF THE INVENTION

An object of the present invention is to provide methods for preventing and treating viral, bacterial or parasite infections, especially of human papillomavirus, and the development of benign or malign consequences of such infections, as well as means for treating and preventing cancer.

The present invention provides for the use of a modified HPV-L1 protein devoid of type-specific epitopes causing production of neutralising antibodies, as a carrier of a substance into cells. As a result of the modification, this HPV-L1 protein carrier does not induce production of overt neutralising antibodies towards the carrier itself. In an embodiment of the invention, one or more amino acids may be deleted from said protein. In particular, the invention provides for such an HPV-L1 protein in fusion with a peptide.

The invention also provides for such a carrier, which is capable of giving rise to a protective antibody response, which antibody response may be cross-reactive towards two or more serologically defined subtypes of human papillomavirus. The carrier must be physically coupled, i.e. fused, to the peptide for which it acts as a carrier, thus creating a fusion protein. Particularly, peptides derived from HPV proteins and defining linear antibody epitopes and T-cell epitopes are recognised. There is also envisaged combinations of said carrier with a minor coat protein of human papillomavirus (HPV-L2 protein), native or modified. Also this HPV-L2 protein can itself be fused to one or more further peptides.

The invention also provides for an oligo- or polynucleotide coding for said carrier. The invention makes it possible to create a better basis for eliciting an MHC class I mediated response, i.e. creating cytotoxic T-cells, without giving rise to type-specific neutralising antibodies towards the carrier, or without type-specific neutralising antibodies being present at the start. It is also possible to use an HPV-L1 protein, modified as described above, as a carrier of oligo- or polynucleotides to cells.

The invention also provides for a recombinant papilloma virus like particle (VLP) for introduction of a peptide into cells, comprising a major capsid protein L1 of human papilloma virus (HPV-L1 protein), wherein the major capsid protein L1 of human papilloma virus has been intentionally modified by substitution of specific amino acids to abolish major type specific epitopes causing production of neutralising antibodies. The L1 protein may be derived from human papilloma virus 16. The L1 protein of HPV 16 may be modified by substitution of two or more of amino acids including, but not limited to, Asn56, Asp138, Asn270, Asn285, Ile348, Ser349, Thr350, Ser351, Glu352, THr353. The peptide may be genetically fused to the L1 protein.

The invention also provides a composition comprising the recombinant papilloma virus like particles (VLP) of the present invention.

### BRIEF DESCRIPTION OF THE DRAWINGS

FIG. 1 shows electron miscroscopy analysis of the ITSE-depleted HPV 16 VLP. FIG. 1A shows ITSE-depleted HPV 16 VLP at 100 nm. FIG. 1B shows ITSE-depleted HPV 16 VLP at 200 nm. FIG. 1C shows ITSE-depleted HPV 16 VLP at 0.2 micrometers.

FIG. 2 shows a graph of OD versus Serum dilution when PBS, wildtype HPV16 VLPs and ITSE-depleted HPV16 VLPs were administered to mice.

### DETAILED DESCRIPTION OF THE INVENTION

In one of its aspects, the invention provides for a carrier for the introduction of a substance into cells, comprising a major capsid protein L1 of human papillomavirus (HPV-L1 protein) which has been intentionally modified to remove type-specific epitope(s) causing production of neutralising antibodies. In one preferred embodiment, the HPV-L1 protein is in fusion with a peptide.

Preferably, the peptide comprises one or more T-cell epitopes, especially epitopes derived from tumor, bacterial, parasite, viral or auto-antigens. In another preferred embodiment, the peptide comprises one or more antibody epitopes, such as tumor, bacterial, parasite, viral or auto-antigens, especially papillomavirus antigens.

The carrier can also be combined with a minor capsid protein L2 of human papillomavirus (HPV-L2 protein), which in its turn may be fused to one or more further peptides. These further peptides are T-cell or antibody epitopes, which may be derived from tumor, bacterial, parasite, viral or auto-antigens.

In a further embodiment, the fusion protein is used as a carrier of oligo- or polynucleotides, such as oligo- or polynucleotides which encode an antigen or an immunostimulatory (poly)peptide.

In another aspect, the invention provides an oligo- or polynucleotide encoding for the carrier as defined.

In further aspects, the invention provides for vaccines, comprising as an active ingredient a carrier or an oligo- or polynucleotide as defined above.

In further aspects, the invention provides methods of preventing or treating viral, bacterial or parasite infections by vaccination with a carrier or an oligo- or polynucleotide as defined above. In a preferred embodiment the infections is caused by papillomavirus. In addition, methods are provided for preventing or treating development of benign or malign consequences of human papillomavirus infection by vaccination with a fusion protein or an oligo- or polynucleotide as defined above.

In embodiments of the methods said human papillomavirus infection is warts or laryngeal papillomatosis. Further aspects of the invention comprise methods of preventing or treating of cancer, including cancer of cervix, penis, vulva, vagina, anus and orofarynx, by vaccination with a fusion protein or an oligo- or polynucleotide as defined above.

### EXAMPLE 1

Upon immunization of a subject with human papillomavirus type 16 wild type (wt) virus-like particles (VLPs), the vast majority of neutralizing antibodies generated are directed against an immunodominant type-specific epitope (ITSE) defined by the monoclonal antibody V5 (9,10). One aspect of the present invention provides for the generation of a human papillomavirus (HPV) type 16 L1 protein which is depleted of the immunodominant type-specific epitope (ITSE), but is still capable of assembling into virus-like particles.

The C-terminal truncated-20 amino acid HPV16 L1 major capsid protein (Ac: af393502.1) was analysed using 3D structural analysis for identification of putative antigenic structures. Three surface exposed hypervariable loops were pinpointed as potential sites for conformational epitopes (11), and amino acids from these loops were included in the mutagenesis strategy. Selected amino acids that were surface exposed and protruding outwards were substituted with GLY, SER or ALA, as it is known in the art that GLY, SER and ALA are uncharged and relatively small amino acids. Substitution with GLY, SER and ALA does not disrupt the 3D structure of HPV16 VLPs, but will affected the conformational epitope.

In addition, all substituted amino acids were variable between closely related HPV-types, and mutations in these sites were not expected to affect the ovarall 3D structure. Thus, the following substitutions were made: aa56 ASN→GLY, aa138 ASP→GLY, aa270 ASN→GLY, aa 285 ASN→SER, aa348 ILE→ALA, aa349 SER→GLY, aa 350 THR→GLY, aa351 SER→GLY, aa352 GLU→GLY and aa 353 THR→GLY, and a ITSE-depleted HPV 16 L1 VLP generated. All mutations in the HPV16 L1 sequence were performed using Stratagene's Quickchange Multi Site-directed Mutagenesis Kit. Constructs were then expressed in a baculovirus/insect cell system and purified as VLPs using CsCl density gradient (12,13,14). It was then shown that the ITSE-depleted HPV16 L1-particle were able to self assemble into VLPs.

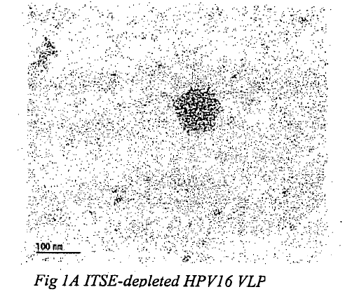

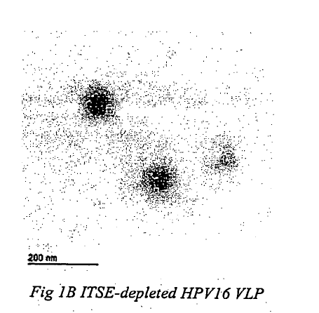

Electron Microscopy confirmed that HPV16 ITSE-depleted L1-protein can self assemble to form virus-like particles. The particles were viewed after direct adsorption onto carbon-coated copper grids. The grids were stained with 0.75% uranyl formate and viewed using a JEOL 200CX transmission electron microscope. Size and purity of the L1 protein was further confirmed by western blot. The expected size of VLPs is 55 nm. The electron microscopy analysis showed that the ITSE-depleted VLPs were able to form fully assembled VLPs, as shown in FIGS. 1A-1C.

The ITSE-depleted HPV16 VLPs were then characterized with antibodies. In order to verify ITSE-depletion and assembly of VLPs, ELISA plates were coated overnight at 4° C. with 50 μl of baculovirus-produced ITSE-depleted HPV16 L1- and wildtype VLPs at a concentration of 10 μg/ml in cold PBS pH 7.2. Disrupted HPV16 ITSE- and wildtype particles were used as negative controls. They were generated through alkaline treatment. The virus-like particles were incubated at room temperature for 4 hours in carbonate buffer pH 9.6, and then the disrupted particles were coated overnight at room temperature.

After the plates were washed four times with PBS-0.5% Tween 20 (PBS-T), they were blocked with PBS supplemented with 10% horse serum (10% HS-PBS) at room temperature for 1 hour. The blocking solution was then discarded and replaced with 50 μl V5 monoclonal antibody or D9 monoclonal antibody (kindly provided by Neils Christensen) diluted in 10% HS-PBS and incubated for 1 hour. Sheep anti-mouse-Ig G horseradish peroxidase conjugate (Amersham) 50 μl (diluted 1:2,000 in 10% HS-PBS) was added to the ELISA plates after washing four times with PBS-T. The plates were incubated for an hour at room temperature and washed four times with PBS-T. 3,3′,5,5′-tetramethyl-benzidine substrate (100 μl per well) (TMB BD Pharmingen) was added and was allowed to react for 15 minutes. The reaction was stopped by the addition of 100 μl of 1 M HCl/well, and the optical density was read at 450 nm.

TABLE 1

| | | D9**(OD₄₅₀) |
|---|---|---|
| | V5*(OD₄₅₀) | Specific for disrupted |
| | Specific for ITSE | VLPs |
| Intact Constructs | | |
| Intact ITSE-depleted VLPs | 0.064 | 0.261 |
| Intact wildtype VLPs | 2.133 | 0.194 |
| Disrupted Constructs | | |
| Disrupted ITSE-depleted | 0.041 | 2.095 |
| VLPs | | |
| Disrupted wildtype VLPs | 0.040 | 2.122 |

*V5 antibody reacts with the immunodominant type-specific epitope exposed on correctly assembled VLPs only. Upon immunization with VLPs the vast majority of neutralizing antibodies generated are directed against this epitope.
**D9 antibody reacts with an epitope exposed on disrupted VLPs but not on intact VLPs

As shown in Table 1, disrupted, but not intact, VLPs reacted with the D9 antibody, indicating correct assembly of both wildtype and mutated VLPs. Intact wild type VLPs were recognized by V5, whereas no significant binding to mutated VLPs was recorded, demonstrating loss of ITSE and consequently reduced antigenicity of mutated VLPs.

Immunization of mice was then performed. C57BL/6 mice were injected subcutaneously with 10 μg of VLPs emulsified in Freund's Complete Adjuvant. Booster injections, consisting of 10 μg VLPs in Freund's Incomplete Adjuvant, were given on day 14 and 28 after the first immunization. Control animals received adjuvant only. Each group (ITSE-depleted VLP-, wild type VLP- and vehicle group) consisted of 5 mice. The mice were bled through vena saphena on day 27 and 35. Blood was collected and centrifuged, serum was collected and stored.

Serum was analyzed for immune response to HPV-16 L1. ELISA plates were coated overnight at 4° C. with 50 μl of baculovirus-produced wildtype HPV-16 L1 VLPs at a concentration of 1 μg/ml. The plates were washed four times with PBS-T and blocked with 10% HS-PBS, and incubated at room temperature for 1 hour. Mouse sera from immunized mice (second bleed) were added to wells at dilutions from 1:300-1:27000 (diluted in 10% HS-PBS) and incubated for 1 hour. After washing, bound antibody was detected using sheep anti-mouse-Ig G horseradish peroxidase conjugate (Amersham, diluted 1:2000 in 10% HS-PBS). The plates were incubated for an hour at room temperature and washed four times with PBS-T. TMB (3,3′,5,5′-tetramethyl-benzidine substrate, BD Pharmingen) 100 μl per well was added, and plates allowed to react in for 15 minutes. The reaction was stopped with the addition of 100 μl of 1 M HCl/well, and the optical density was read at 450 nm.

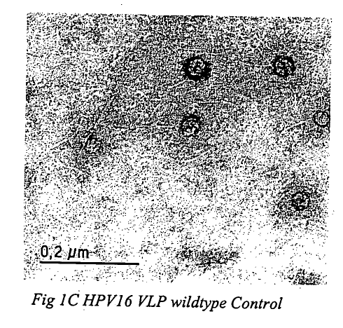

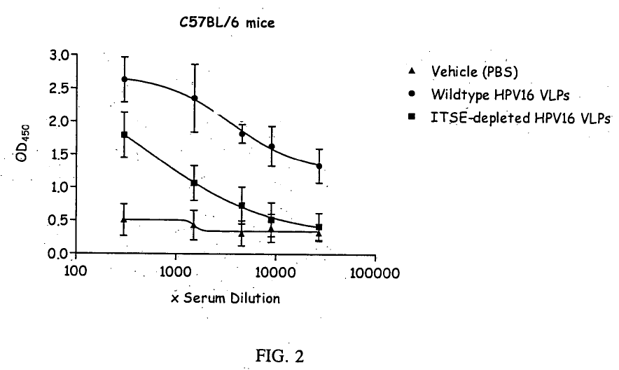

As shown in FIG. 2, ELISA values represent the means (+/−standard deviation) of OD-values for individual mice within each immunization group. Each immunization group consisted of 5 mice. Anti-HPV titers were defined as the dilution that yielded an OD value of 1.5. Thus, the anti-HPV titers of sera from mice immunised with wild type and ITSE-depleted HPV 16 VLPs were 11200 and 540 respectively.

These mutations resulted in a protein capable of self-assembling into VLP particles that were considerably (20 times) less immunogenic than wild type HPV 16 VLPs. There is no evidence that a carrier according to the invention will lead to the production of neutralizing antibodies.

All references discussed above are herein incorporated by reference in their entirety for all purposes. While this invention has been particularly shown and described with references to preferred embodiments thereof, it will be understood by those skilled in the art that various changes in form and details may be made therein without departing from the spirit and scope of the invention as defined by the appended claims.

### REFERENCES

1. H. zur Hausen, Viruses in human cancers, Science (1991) 254:1167-1173.

2. D. R. Lowy and J. T. Schiller. Papillomaviruses and cervical cancer: Pathogenesis and vaccine development, J. Natl. Cancer Inst. Monographs (1998) 23: 27-30.

3. W. I. White, S. D. Wilson, W. Bonnez, R. C. Rose, S. Koenig and J. A. Suzich' In vitro infection and type-restricted antibody-mediated neutralization of authentic human papillomavirus type 16, J. Virol. (1998) 72: 959-964.

4. H. L. Greenstone, J. D. Nieland, K. E. deVisser, M. E. De Bruijn, R. Kirnbauer, R. B. Roden, D. R. Lowy, W. M. Kast and J. T. Schiller' Chimeric papillomavirus virus-like particles elicit antitumor immunity against the E7 oncoprotein in an HPV16 tumor model, Proc. Natl. Acad. Sci. USA (1998) 95: 1800-1805.

5. S. Peng, I. H. Frazer, G. J. Fernando and J. Zhou, Papillomavirus virus-like particles can deliver defined CTL epitopes to the MHC class I pathway, Virology (1998) 240: 147-157.

6. M. Muller, J. Zhou, T. D. Reed, C. Rittmuller, A. Burger, J. Gabelsberger, J. Braspenning and L. Gissmann, Chimeric papillomavirus-like particles, Virology (1997) 234: 93-111.

7. White, W. I., Wilson, S. D., Palmer-Hill, F. J., Woods, R. M., Ghim, S.-J., Hewitt, L. A., Goldman, D. M., Burke, S. J., Jenson, A. B., Koenig, S. and Suzich, J. A., Characterization of a Major Neutralizing Epitope on Human Papillomavirus Type 16 L1, Virology (1999), 73:4882-4889.

8. Wang, Z., Christensen, N. D., Schiller, J. T. and Dillner, J., A monoclonal antibody against intact Human Papillomavirus type 16 capsids blocks the serological reactivity of most human sera, J. Gen. Virol. (1997) 78:2209-2215.

9. Neil D. Christensen, Nancy M. Cladel, Cynthia A. Reed, Lynn R. Budgeon, Monica E. Embers, Dee M. Skulsky, William L. McClements, Steven W. Ludmerer and Kathrin U. Jansen, Hybrid papillomavirus L1 molcules assemble into virus-like particles that reconstitute conformational epitopes and induce neutralising antibodies to distinct HPV types, Virology (2001) 291, 324-334.

10. Zhaohui Wang, Neil Christensen, John T. Shiller and Joakim Dillner, A monoclonal antibody against human papillomavirus type 16 capsids blocks the serologickal reactivity of most human sera, J. Gen. Virol. (1997) 78:2209-2215.

11. Xiaojiang S. Chen, Robert L. Garcea, Ilya Goldberg, Gregory Casini and Stephen C. Harrison, Structure of Small Virus-like Particles Assembled from the L1 Protein of Human Papillomavirus 16, Mol Cell, (2000) 5: 557-567.

12. P. Le Cann, P. Coursaget, S Iochmann and A Touze, Self-assembly of human papillomavirus type 16 capsids by expression of the L1-protein in insect cells, FEMS Microbiol. Lett. (1994) 117:269-274.

13. A. Touze, S. El Mehdaoui, P-Y. Sizaret, C. Mougin, N. Nunoz and P. Coursaget, The L1 major capsid protein of human papillomavirus type 16 variants affect the yields of virus-like particle produced in a insect cell expression system, J. Clin. Microbiol., (1998) 36: 2046-2051.

14. N. D. Christensen R. Höpfl. S. L. DiAngelo, N. M. Cladel, S. D. Patric, P. A.

15. Welch, L. R. Budgeon, C. A. Reed and J. W. Kreider, Assembled baculovirus-expressed HPV-11 L1 virus-like particles are recognized by neutralizing monoclonal antibodies and induce high titers of neutralising antibodies, J. Gen. Virol. (1994) 75: 2271-2276.

1. A recombinant papilloma virus like particle (VLP) for introduction of a peptide into cells, comprising a major capsid protein L1 of human papilloma virus (HPV-L1 protein) wherein the major capsid protein L1 of human papilloma virus has been intentionally modified by substitution of specific amino acids to abolish major type specific epitopes causing production of neutralising antibodies.

2. The recombinant papilloma virus like particle (VLP) according to claim 1, wherein the L1 protein is derived from human papilloma virus 16.

3. The recombinant papilloma virus like particle (VLP) according to claim 2, wherein the L1 protein of HPV 16 has been modified by substitution of two or more of amino acids selected from the group consisting of Asn56, Asp138, Asn270, Asn285, Ile348, Ser349, Thr350, Ser351, Glu352, THr353.

4. The recombinant papilloma virus like particle (VLP) according to claim 1, wherein the peptide is genetically fused to the L1 protein.

5. A composition comprising the recombinant papilloma virus like particle (VLP) of claim 1.
